(19) United States
(12) Patent Application Publication
(10) Pub. No.: US 2005/0065369 A1
(43) Pub. Date: Mar. 24, 2005

(54) Preparation of polyaryl carboxylic acids

(76) Inventors: Zhenrong Qian, Bridgewater, NJ (US); Heng Eric Su, Marlboro, NJ (US); Lee Alan DeWitt, Howell, MI (US)

Correspondence Address: Synnestvedt & Lechner LLP;2600 Aramark Tower, 1101 Market Street, Philadelphia, PA, 19107-2950, US

(21) Appl. No.: 10/082,996

(22) Filed: Feb. 26, 2002

Related U.S. Application Data
(60) Provisional application No. 60/271,559, filed on Feb. 26, 2001.

(51) Int. Cl. C07C 63/14; C07C 63/307; C07C 63/313

(52) U.S. Cl. 562/480

(57) ABSTRACT

A method for the preparation of an polyaromatic carboxylic acid compound and/or sale thereof comprising reacting an aromatic boronic acid with a halo-substituted, aromatic carboxylic acid compound and/or sale thereof.

### FIELD OF INVENTION

The present invention relates to processes useful in the preparation of polyaryl compounds, and more particularly to the preparation of compounds useful in the preparation of compounds having pharmaceutical applications.

The field of pharmaceutical discovery increasingly requires the development of new and improved methods for the preparation of intermediate compounds using in preparing those compounds that are effective in the treatment of the myriad of ailments that afflict humans and animals alike. Included in these compounds are the polyaryl compounds that have been found to have a variety of pharmaceutical applications including applications as anti-fungal agents, more specifically as anti-fungal agents useful against such microorganisms as Candida albicans. See. U.S. Pat. No. 5,965,525 (Burkhardt, et al. ) which discloses polyaromatic acylated microbially based cyclic peptides, prepared from an activated polyaromatic carboxylic acid intermediate, with enhanced potency against pathogenic strains such as Candida albicans. The preparation of these anti-fungal compounds have been facilitated by the use of carboxylic acid intermediates that have been found advantageous for coupling to active amino groups on proteins and polypeptides.

### REPORTED DEVELOPMENTS

Substituted polyaryl compounds have been prepared by several different cross-coupling type reactions in which rings are joined through the formation of new carbon-carbon bonds. These well-known cross-coupling reactions are useful in the synthesis of a broad scope of biaryl, polyaryl, and polyheteroaryl compounds.

Depending on the chemical structure of the starting materials, cross-coupling reactions lead to either symmetrical or unsymmetrical polyaryls. As an undesirable side reaction, starting materials may self-couple leading to the formation of impurities which may be difficult and costly to remove from the cross-coupled product. It is therefore desirable to find alternate methods that optimize the yield of the cross-coupled product and simplify purification procedures.

As mentioned above, polyaryl carboxylic acid compounds have been used in the prior art as intermediates in the synthesis of anti-fungal agents. These polyaryl carboxylic acid compounds have been prepared directly through the cross-coupling of the magnesium halide salt of a halo-aromatic carboxylic acid with an appropriately substituted aromatic Grignard reagent in the presence of a nickel or palladium catalyst. The drawback to this method is that each starting compound has a tendency to self-couple leading to the formation of undesirable impurities.

The “Suzuki” coupling reaction was first reported in the literature in 1981 Suzuki et al disclosed the palladium-catalyzed formation of biaryl compounds by cross-coupling phenylboronic acids with haloarenes. Miyaura, N, Yanagi, T, Suziki, A: Synth. Commun. 1981, 11, 513. The cross-coupling reaction was conducted in refluxing benzene or toluene in the presence of a base such as aqueous NaOH and Na2CO3. Haloarene substituents disclosed included methyl, methoxy, among others, but not carboxylic acid. In 1992, Suzuki et al extended the scope of the reaction by reporting on modified coupling reaction conditions consisting of the use of K3PO4 in DMF in combination with the trimethylene glycol ester of the arylboronic acid. These modified conditions were found to be effective with boronic ester compounds substituted with electron-withdrawing substituent groups, such as formyl groups, which, in the absence of the protecting boronic ester, tend to accelerate competitive hydrolytic deboration. Watanabe, T., Miyaura, N., Suzuki, A., Synlett, 1992, 207.

The Suzuki reaction was applied more recently as disclosed in U.S. Pat. No. 5,965,525 (Burkhardt, et al.) where the inventors prepared a pharmaceutical intermediate by Suzuki coupling a series of 4-alkoxy and 4-alkoxyalkoxy biphenyl boronic acids with methyl 4-iodo benzoate. The resulting methyl carboxylic ester was hydrolyzed to yield the free acid which was converted into the 2,4,5, trichlorophenyl ester used to N-acylate the free amino groups of a microbially produced cyclic peptide. The resulting amide is reported to exhibit enhanced potency against pathogenic strains such as Candida albicans.

Ennis et al, Org. Pros Res. Chem. (1999), 3 (4), 248-252, reported using the Suzuki coupling reaction to prepare biphenyl carboxylic acids , useful as key intermediates of anti-depression pharmaceuticals, by reacting a brominated phenyl compound with a carboxyl substituted phenylboronic acid. These reactions were conducted in aqueous media and produced products contaminated with from 6 to 80 ppm of the palladium catalyst.

Despite the variety of substituted polyaryl compounds reported to have been successfully coupled using the Suzuki reaction, coupling of boronic acid compounds with halo-substituted aromatic carboxylic acids or their salts has not been reported. Accordingly the scope of the polyaryl compounds prepared by the Suzuki coupling has been limited.

### SUMMARY OF THE INVENTION

The present invention relates to a method for the preparation of a polyaromatic carboxylic acid and/or salt thereof comprising reacting an aromatic boronic acid with a halo-substituted, aromatic carboxylic acid and/or salt thereof.

A preferred aspect of the present invention is a method for preparing carboxyl substituted polyaryl compounds of formula I, and/or salts thereof,
R₁-A₁-(A₁)_{Y-1}-(A₂)_{X-1}-A₂-COOH (I)
by cross-coupling an aromatic boronic acid or borate of formula II
R₁-A₁-(A₁)_{Y1}-B(OR)₂ (II)
with a halo-substituted aromatic carboxylic acid of formula III, and/or salts thereof,
halo-(A₂)_{X-1}-A₂-COOH (III)
wherein:

R is hydrogen, lower alkyl or alkylene, which forms a cyclic boronic acetal;

R₁ is independently hydrogen or a substituent group;

A₁ and A₂ are each independently a substituted or unsubstituted monocyclic or polycyclic aromatic groups; and

X and Y are independently 1 to about 10.

The present method is a surprising improvement in the prior methods for preparing polyaromatic carboxylic acids, the improvement comprising reacting a free carboxylic acid substituted aryl intermediate and/or a salt thereof with an appropriately substituted aromatic boronic acid. The application of the boronic coupling reaction to an unprotected carboxylic intermediate permits the elimination of the required de-protective hydrolysis disclosed in the prior art. Furthermore, the present method results in easier isolation of the carboxylic product, and in good yield substantially free of difficult to remove by-products.

Further aspects and advantages of the present invention are described in more detail in the following section.

### DETAILED DESCRIPTION OF THE INVENTION

The present invention comprises a method that couples organic compounds characterized as “aromatic” or “aryl” which signify a cyclic planar structure, or ring, wherein each atom of the ring or cycle has a p orbital which is perpendicular to the plane of the ring; a single aromatic ring must contain a total of paired pi electrons equal to 4n+2, where n is an integer.

Aromatic compounds are classified as monocyclic, polycyclic, and heterocyclic depending on the number of rings, and the inclusion of atoms other than carbon making up the cyclic ring structure. Preferred examples of aryl radicals include phenyl, biphenyl, triphenyl, o-tolyl, 4-methoxyphenyl, 2-(tert-butoxy)phenyl, 3-methyl-4-methoxyphenyl, 2-CF₃-phenyl, 2-fluorophenyl, 2-chlorophenyl, 3-nitrophenyl, 3-aminophenyl, 3-acetamidophenyl, 2-amino-3-(aminomethyl)phenyl, 6-methyl-3-acetamidophenyl, 6-methyl-2-aminophenyl, 6-methyl-2,3-diaminophenyl, 2-amino-3-methylphenyl, 4,6-dimethyl-2-aminophenyl, 4-hydroxyphenyl, 3-methyl-4-hydroxyphenyl, 4-(2-methoxyphenyl)phenyl, 2-amino-1-naphthyl, 2-naphthyl, 3-amino-2-naphthyl, 1-methyl-3-amino-2-naphthyl, 2,3-diamino-1-naphthyl, 4,8-dimethoxy-2-naphthyl. Each of the foregoing groups may also be linked para to another phenylene group and may be optionally substituted with one or more substituents. As used herein, “substituted” is intended to indicate that one or more hydrogens on the atom indicated in the expression using “substituted” is replaced with a selection from the indicated “substituent” group(s), provided that the indicated atom's normal valency is not exceeded, and that the substitution results in a stable compound. Exemplary substituents include alkyl, alkoxy, alkenyl, halogen, hydroxy, amino, azido, nitro, cyano, haloalkyl, carboxy, alkoxycarbonyl, cycloalkyl, cycloalkenyl, alkanoylamino, amido, amidino, alkoxycarbonylamino, N-alkylamidino, alkylamino, dialkylamino, aminoalkyl, alkylaminoalkyl, dialkylaminoalkyl, N-alkylamido, N,N-dialkylamido, aralkoxycarbonylamino, alkylthio, alkylsulfinyl, alkylsulfonyl, oxo and the like.

The present invention more particularly concerns the preparation of “polyaromatic” or “polyaryl” compounds, which describe compounds, comprised of more than one aromatic ring structure connected by chemical bonds between ring carbon atoms. These multi-ring structures may be bonded by a single carbon-carbon bond, resulting in for example, polyphenyl structures, or bonded by two carbon-carbon bonds resulting in fused ring structures. Many such fused ring system may be described by the term, “benzo”, which, alone or in combination, means the divalent radical C6 H4 derived from benzene. “benzo fused” forms a ring system in which benzene and a cycloalkyl or aryl group have two carbons in common, for example tetrahydronaphthylene and the like. In the description of the present invention the term, “bicyclic” is intended to include both fused ring systems, such as naphthyl and beta.-carbolinyl, and the single bonded polycyclic ring systems, such as biphenyl, phenylpyridyl and diphenylpiperazinyl. The polycyclic aromatic ring systems are the result of the coupling reaction of the present invention.

A more generic term to describe rings systems used in the present invention, as substituents groups, is “carbocyclic radical”, which describes radicals derived from a saturated or unsaturated, substituted or unsubstituted 5 to 14 member organic nucleus whose ring forming atoms (other than hydrogen) are solely carbon atoms. Typical carbocyclic radicals are cycloalkyl, cycloalkenyl, phenyl, naphthyl, norbornanyl, bicycloheptadienyl, tolulyl, xylenyl, indenyl, stilbenyl, terphenylyl, diphenylethylenyl, phenylcyclohexyl, acenaphthylenyl, and anthracenyl, biphenyl, bibenzylyl and related bibenzylyl homologues. octahydronaphthyl, tetrahydronaphthyl, octahydroquinolinyl, dimethoxytetrahydronaphthyl, 2,3-dihydro-1H-indenyl, azabicyclo[3.2.1]octyl ad the like. The term “cycloalkyl”, alone or in combination, means a saturated monocyclic hydrocarbon radical. Preferred groups contain about 5 to about 12 carbon atoms, more preferably about 5 about 10 carbon atoms, even more preferably a bout 5 to about 7 carbon atoms, and which is optionally substituted as defined herein with respect to the definition of aryl. Examples of such cycloalkyl radicals include cyclopentyl, cyclohexyl, dihydroxycyclohexyl, ethylenedioxycyclohexyl, cycloheptyl, and the like. Similar to the previous term, “cycloalkenyl”, alone or in combination, means a partially unsaturated, preferably one double bond, monocyclic hydrocarbon radical. Preferred groups contain about 5 to about 12 carbon atoms, more preferably about 5 about 10 carbon atoms, even more preferably about 5 to about 7 carbon atoms, and which is optionally substituted as defined herein with respect to the definition of aryl. Examples of such cycloalkenyl radicals include cyclopentenyl, cyclohexenyl, dihydroxycyclohexenyl, ethylenedioxycyclohexenyl, cycloheptenyl, and the like.

When the carbon-containing ring also includes a heteroatom, such as nitrogen, oxygen and sulfur, the term “heterocycle” is used. More particularly, heterocycle means a stable 5- to 6-membered monocyclic ring, which is saturated, partially unsaturated, or aromatic, and which consists of carbon atoms and from 1 to about 3 heteroatoms independently selected from the group consisting of N, O and S. The nitrogen and sulfur heteroatoms may optionally be oxidized. The heterocyclic ring may be attached to its pendant group at any heteroatom or carbon atom, which results in a stable structure. The heterocyclic rings described herein may be substituted on carbon or on a nitrogen atom if the resulting compound is stable. If specifically noted, the nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. As used herein, the term “aromatic heterocyclic system” is intended to mean a stable 5- to 6-membered monocyclic heterocyclic aromatic ring which consists of carbon atoms and from 1 to 3 heteroatoms independently selected from the group consisting of N, O and S. It is preferred that the total number of S and O atoms in the aromatic heterocycle is not more than 1. Examples of heterocycles include, but are not limited to, anthranilyl, azaindolyl, benzofuranyl, 1,2-benzisoxazolyl, benzopyranyl, benzoxazolyl, benzothiazolyl, benzotriazolyl, benzylpyridinyl, dibenzofuranyl, 4-benzyl-piperazin-1-yl, carbazolyl, 2,3-dihydrobenzofuryl, dibenzothiophenyl, 2,3-dihydroindolyl, ethylenedioxyphenyl, 6H-1,2,5-thiadiazinyl, 2H,6H-1,5,2-dithiazinyl, furanyl, furazanyl, imidazolidinyl, imidazolinyl, imidazolyl, imidazo(1.2-A)pyridinyl, indolyl, indazolyl, isoxazolyl, methylenedioxyphenyl, morpholinyl, norharmanyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, pyrazolidonyl, pyridazinonyl, pyrrolidonyl, phthalazinyl, phenylimidazolyl, piperazinyl, piperidinyl, pteridinyl, piperidonyl, 4-piperidonyl, piperazinyl, pteridinyl, purinyl, pyranyl, pyrazinyl, pyrazolidinyl, pyrazolinyl, pyrazolyl, pyridazinyl, pyridinyl, pyridyl, dipyridylyl. phenylpyridinyl, pyrimidinyl, phenylpyrimidinyl, pyrrolidinyl, 2-pyrrolidonyl, 2H-pyrrolyl, 4-piperidonyl, pyrrolinyl, pyrrolyl, quinolinyl, quinazolinyl, quinoxalinyl, tetrahydrofuranyl, tetrahydroquinolinyl, 1,2,3,4-tetrahydroisoquinolinyl, 1,2,3,4- tetrahydro- 1-oxo-isoquinolinyl, tetrahydrothienyl and its sulfoxide and sulfone derivatives, 6H-1,2,5-thiadiazinyl, 1,2,3-thiadiazolyl, 1,2,4-thiadiazolyl, 1,2,5-thiadiazolyl, 1,3,4-thiadiazolyl, thiamorpholinyl, thianaphtheneyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, and 1,3,4-triazolyl. Preferred heterocycles include, but are not limited to, pyridinyl, furanyl, thienyl, pyrrolyl, pyrazolyl, imidazolyl, and oxazolidinyl. Also included are fused ring and spiro compounds containing, for example, the above heterocycles. The fused rings may be described as “heterocyclo fused” and form a ring system in which a heterocyclyl or heteroaryl group of 5-6 ring members and a cycloalkyl or aryl group have two carbons in common. Examples include indole, isoquinoline, tetrahydroquinoline, and methylenedioxybenzene.

The classes of heteroatom-containing rings that are also aromatic in character are described as “heteroaryl”. Such heteroaryl groups signify a monocyclic or bicyclic, aromatic heterocycle radical. Preferred heteroaryl include at least one, preferably 1 to about 4, more preferably 1 to about 3, even more preferably 1 to 2, nitrogen, oxygen or sulfur atom ring members. More preferred heteroaryl radicals include preferably 5 to about 6 ring members in each ring, which is optionally saturated carbocyclic fused, preferably 3 to 4 carbon atoms to form 5 to 6 ring member rings and which is optionally substituted as defined above with respect to the definitions of aryl. The most preferred radicals are monocyclic. Examples of such heteroaryl groups include thienyl, furyl oxazolyl, thiazolyl, benzothiazolyl, benzofuryl, benzothienyl, imidazolyl, pyrrolyl, pyrazolyl, pyridyl, 3-(2-methyl)pyridyl, 3-(4-trifluoromethyl)pyridyl, pyrimidyl, 5-(4-trifluoromethyl)pyrimidyl, pyrazinyl, triazolyl, indolyl, quinolinyl, 5,6,7,8-tetrahydroquinolyl, 5,6,7,8-tetrahydroisoquinolinyl, quinoxalinyl, benzimidazolyl, and benzoxazolyl. Similarly, the terms “heteroaralkyl” and “heteroarylalkyl,” alone or in combination, means an alkyl radical as defined above in which at least one hydrogen atom, preferably 1 to 2, is replaced by a heteroaryl radical as defined above. Examples include 3-furylpropyl, 2-pyrrolyl propyl, chloroquinolinylmethyl, 2-thienylethyl, pyridylmethyl, 1-imidazolylethyl and the like.

The present method uses intermediates and produces products containing an “acidic or acid group”, which in the broadest sense means an group that acts as a proton donor capable of hydrogen bonding. In general, acid groups soluble in aqueous systems include sodium bisulfate, potassium bisulfate, ammonium chloride, lithium bisulfate and the like, while “strong acid” refers to any acid having a pKa less than 4.7, which include, but are not limited to mineral acids such as hydrochloric acid, hydrobromic acid, hydroiodic acid, sulfuric acid, nitric acid, phosphoric acid; and organic acids such as formic acid, acetic acid, methanesulfonic acid, trifluoroacetic acid, propionic acid, butyric acid, valeric acid, caproic acid, oxalic acid, benzenesulfonic acid, and p-toluenesulfonic acid.

The present invention uses and produces acid compounds more specifically characterized as a “carboxylic acid” which means a compound containing a functional group described by the formula, —C(O)—OH. A related class of compounds including the closely related functional group “carboxy”, described by the formula, —C(O)—O—, is described as “acyloxy”, which means a hydrocarbon carboxy radical group. Examples of acyloxy groups include arylcarboxy groups and alkylcarboxy radicals containing from one to about 13 carbon atoms. More preferred aliphatic groups include alkanoyloxy groups having about 2 to about 6 carbon atoms. Exemplary groups include acetyloxy, propionyloxy, butyryloxy and isobutyryloxy. Esterified carboxyl groups include, for example, alkoxycarbonyl group, aralkyloxycarbonyl group and aryloxycarbonyl group, defined hereinbelow. A further related class of compounds including the carbonyl “—C(O)—” functionality is “alkanoyl”, which alone or in combination, means a radical of the type “R—C(O)—” wherein “R” is an alkyl radical as defined above and is. Examples of such alkanoyl radicals include acetyl, trifluoroacetyl, hydroxyacetyl, propionyl, butyryl, valeryl, 4-methylvaleryl, and the like.

The present invention may, in lieu of the aromatic acid use its “salt” which means a chemical compound characterized by a cation-anion pair associated by an ionic bond. Salts are well known by those skilled in the art, and are generally prepared by reacting the free base or acid with stoichiometric amounts or with an excess of the desired salt-forming acid or base in a suitable solvent or various combinations of solvents. The salts described herein relate principally to the basic salts of organic acids, including the carboxylic acids used in the method of the present invention. When intermediates or final compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al, J. Pharm. Sci. 66, 1 (1977). A “pharmaceutically acceptable salt” refers to derivatives of the disclosed compounds wherein the intermediates or final compound are modified by making acid or base salts thereof using complementary metal and/or amine bases known to be used in the pharmaceutical arts. Examples of pharmaceutically acceptable salts include, but are not limited to, alkali or organic salts of acidic residues such as carboxylic acids. The salt's positively charged ionic partner for the negative charge of carboxylic acid of the present invention comprises a “cation” or “positive counter-ion”. Examples of suitable counter ions include metals, but are not limited to positively charged ions or complexes of lithium, sodium, potassium; copper and any salts thereof, such as chloride, bromide or iodide; magnesium and any salts thereof, such as chloride, bromide or iodide; zinc and any salts thereof, such as chloride or bromide; cerium and any salts thereof, such as chloride or bromide; and calcium and any salts thereof, such as chloride or bromide. Examples of positively charged ions or complexes include ammonium and quaternary amines, Li+, Na+, K+, MgCl+, MgBr+, MgI+, ZnCl+, ZnBr+, CaCl+, CaBr+, CeCl.sub.2+, CeBr.sub.2+, CuBr+, and CuCl+.

The following terms are used herein to describe more particular aspects underlying the scope of the present invention.

“Alkyl”, alone or in combination or as part of another substituent, means a straight chain or branched-chain saturated aliphatic monovalent hydrocarbon radical. Alkyl preferably contains 1 to about 15 carbon atoms, more preferably 1 to about 8 carbon atoms, even more preferably 1 to about 6 carbon atoms, yet more preferably 1 to about 4 carbon atoms, still more preferably 1 to about 3 carbon atoms, and most preferably 1 to2 carbon atoms. Examples of alkyl include methyl, ethyl, n-propyl, isopropyl, n-butyl, isobutyl, sec-butyl, tert-butyl, n-pentyl, iso-amyl, hexyl, octyl and the like.

“Alkenyl” employed alone or in combination with other terms means a straight chain or branched monovalent aliphatic hydrocarbon chain and one or more unsaturated carbon-carbon bonds which may occur in any stable point along the chain having the stated number range of carbon atoms. Preferred alkenyl groups include one to about two double bonds, and contain about 2 to about 15 carbon atoms. More preferred alkenyl groups include about 2 to about 8 carbon atoms, and even more preferably about 2 to about 6 carbon atoms, yet more preferably about 2 to about 4 carbon atoms, and still more preferably about 2 to about 3 carbon atoms. Alkenyl groups include for example vinyl, propenyl, crotonyl, isopentenyl, 2-methylpropenyl, 1,4-butadienyl and butenyl isomers.

“Alkynyl” means an aliphatic hydrocarbon chain of either a straight or branched configuration and one or more triple carbon-carbon bonds that may occur in any stable point along the chain. Examples include ethynyl, propynyl and the like.

“Alkoxy” represents an alkyl group as defined above with the indicated number of carbon atoms attached through oxygen. Examples of alkoxy include, but are not limited to, methoxy, ethoxy, n-propoxy, i-propoxy, n-butoxy, s-butoxy, t-butoxy, n-pentoxy, and s-pentoxy.

“Alkenyloxy” represents an alkenyl group as defined above with the indicated number of carbon atoms attached through an oxygen Preferable examples of the alkenyloxy group include two to about ten carbon atoms. Examples include allyloxy, crotyloxy, 2-pentenyloxy and 3-hexenyloxy. Preferable examples of the cycloalkenyloxy group include about three to about ten carbon atoms, such as 2-cyclopentenyloxy and 2-cyclohexenyloxy.

“Alkoxycarbonyl”, alone or in combination, means a radical of the type “R—O—C(O)—” wherein “R—O—” is an alkoxy radical as defined above and “C(O)” is a carbonyl radical. Preferred alkoxycarbonyl groups include about 2 to about five carbon atoms. Examples include methoxycarbonyl, ethoxycarbonyl, propoxycarbonyl and butoxycarbonyl.

“Alkoxycarbonylamino”, alone or in combination, means a radical of the type “R—O—C(O)—NH—” wherein “R—O—C(O)” is an alkoxycarbonyl radical as defined above, wherein the amino racical may optionally be substituted. Exemplary substituents include alkyl, aryl, aralkyl, cycloalkyl, cycloalkylalkyl and the like.

“Alkanoylamino”, alone or in combination, means a radical of the type “R—C(O)—NH—” wherein “R—C(O)—” is an alkanoyl radical as defined above, wherein the amino radical may optionally be substituted. Exemplary substituents include alkyl, aryl, aralkyl, cycloalkyl, cycloalkylalkyl and the like.

“Alkylsulfinyl”, alone or in combination, means a radical of the type “R—S(O)—” wherein “R” is an alkyl radical as defined above and “S(O)” is a mono-oxygenated sulfur atom. Examples of such alkylsulfinyl radicals include methylsulfinyl, ethylsulfinyl, n-propylsulfinyl, isopropylsulfinyl, n-butylsulfinyl, iso-butylsulfinyl, sec-butylsulfinyl, tert-butylsulfinyl and the like.

“Alkylsulfonyl”, alone or in combination, means a radical of the type “R—S(O).sub.2—” wherein “R” is an alkyl radical as defined above and “S(O).sub.2 ” is a di-oxygenated sulfur atom. Examples of such alkylsulfonyl radicals include methylsulfonyl, ethylsulfonyl, n-propylsulfonyl, isopropylsulfonyl, n-butylsulfonyl, iso-butylsulfonyl, sec-butylsulfonyl, tert-butylsulfonyl and the like.

“Alkylthio”, alone or in combination, means a radical of the type “R—S—” wherein “R” is an alkyl radical as defined above and “S” is a sulfur atom. Preferred alkylthio groups include about one to about ten carbon atoms. Examples of such alkylthio radicals include methylthio, ethylthio, n-propylthio, isopropylthio, n-butylthio, iso-butylthio, sec-butylthio, tert-butylthio pentylthio, isopentylthio, neopentylthio, hexylthio, heptylthio and nonylthio and the like.

“Alkenylthio”, alone or in combination, means a radical of the type “R—S—” wherein “R” is an alkenyl radical as defined above and “S” is a sulfur atom Preferred alkenylthio groups include about 2 to about 10 carbon atoms. Examples include allylthio, crotylthio, 2-pentenylthio and 3-hexenylthio.

“Cycloalkenylthio”, alone or in combination, means a radical of the type “R—S—” wherein “R” is an cycloalkenyl radical as defined above and “S” is a sulfur atom Preferred cycloalkenylthio groups include about 3 to about 10 carbon atoms. Examples include 2-cyclopentenylthio and 2-cyclohexenylthio.

“Aralkyl” and “arylalkyl”, alone or in combination, means an alkyl radical as defined above in which at least one hydrogen atom, preferably 1 to 2, is replaced by an aryl radical as defined above. Preferred examples include benzyl, 1-, 2-phenylethyl, dibenzylmethyl, hydroxyphenylmethyl, methylphenylmethyl, diphenylmethyl, dichlorophenylmethyl, 4-methoxyphenylmethyl and the like. For example, phenylmethyl means a methylene diradical substituted with a phenyl radical, i.e., Ph—CH2—, whereas a methylphenyl means a phenylene diradical substituted with a methyl radical, i.e., CH3—Ph—.

“Aralkoxyl”, alone or in combination, means an alkoxy radical as defined above in which at least one hydrogen atom, preferably 1 to 2, is replaced by an aryl radical as defined above. Preferred examples include benzyloxy, 1-, 2-phenylethoxy, dibenzylmethoxy, hydroxyphenylmethoxy, methylphenylmethoxy, dichlorophenylmethoxy, 4-methoxyphenylmethoxy and the like.

“Aryloxy”, alone or in combination, means an aryl radical as defined above in which at least one hydrogen atom, is replaced by an oxygen atom. Preferred aryloxy groups include about 6 to about 14 carbon atoms. Preferred examples include phenoxy, naphthyloxy, toluenoxy, hydroxyphenyoxy, methylphenyloxy, dichlorophenyloxy, 4-methoxyphenyloxy, 4-methoxyphenyl-4-phenoxy, 4-chlorophenoxy and the like.

“Aralkoxycarbonyl”, alone or in combination, means a radical of the type “R—O—C(O)—” wherein “R—O—” is an aralkoxy radical as defined above and “—C(O)—” is a carbonyl radical. Preferred aralkyloxycarbonyl groups include about 8 to about ten carbon atoms. Examples include benzyloxycarbonyl.

“Aryloxycarbonyl”, alone or in combination, means a radical of the tye “R—O—C(O)—” wherein “R—O—” is an aryloxy radical as defined above and “—C(O)—” is a carbonyl radical. Preferred aryloxycarbonyl groups include about seven to about 15 carbon atoms. Most preferred aryloxycarbonyl groups include about 8 to about ten carbon atoms. Examples include phenoxycarbonyl and p-tolyloxycarbonyl.

“Cycloalkylthio”, alone or in combination, means a radical of the type “R—S—” wherein “R” is an cycloalkyl radical as defined above and “S” is a sulfur atom Preferred cycloalkylthio groups include about 3 to about 10 carbon atoms. Examples include cycloalkylthio groups such as cyclobutylthio, cyclopentylthio and cyclohexylthio.

“Aralkylthio”, alone or in combination, means a radical of the type “R—S—” wherein “R” is an aralkyl radical as defined above and “S” is a sulfur atom Preferred aralkylthio groups include about 7 to about 10 carbon atoms. Examples include phenylalkylthio, more specifically for example, benzylthio and phenethylthio.

“Acylthio”, alone or in combination, means a radical of the type “R—S—” wherein “R” is an acyl radical as defined above and “S” is a sulfur atom Preferred acylthio groups include 2 to about 3 carbon atoms. Examples include alkanoylthio groups such as for example acetylthio, propionylthio, butyrylthio and isobutyrylthio.

“Arylthio”, alone or in combination, means a radical of the type “R—S—” wherein “R” is an aryl radical as defined above and “S” is a sulfur atom Preferred arylthio groups include about 6 to about 14 carbon atoms. Examples include phenylthio and naphthylthio. The arylthio group may optionally have one or two substituents such as halogen atom, examples of which include 4-chlorophenylthio.

“Amine” or “amino” means primary, secondary and tertiary amines.

“Aminocarbonyl”, alone or in combination, means an amino substituted carbonyl (carbamoyl) radical, wherein the amino radical may optionally be mono- or di-substituted. Examples of preferred substituents include alkyl, aryl, aralkyl, cycloalkyl, cycloalkylalkyl, alkanoyl, alkoxycarbonyl, aralkoxycarbonyl and the like.

“Aminosulfonyl”, alone or in combination, means an amino substituted sulfonyl radical.

“Halogen” and “halo”, alone or in combination, means fluoro, chloro, bromo or iodo radicals.

“Haloalkyl” means both branched and straight chain saturated aliphatic hydrocarbon groups having the specified number of carbon atoms, substituted with 1 or more halogen. Examples of haloalkyl include, but are not limited to, trifluoromethyl, trichloromethyl, pentafluoroethyl, 1,1,1 -trifluoroethyl, chloromethyl, 1-bromoethyl, fluoromethyl, difluoromethyl, bis(trifluoromethyl)methyl and pentachloroethyl.

“Hydroxyalkyl”, alone or in combination, means an alkyl radical as defined above wherein at least one hydrogen radical is replaced with a hydroxyl radical. Preferred groups replace 1 to about 3 hydrogen by hydroxyl radicals, more preferred replace 1 to about 2 hydrogen by hydroxyl radicals, and most preferred replace one hydrogen radical by a hydroxyl radical. Examples of such radicals include hydroxymethyl, 1-, 2-hydroxyethyl, 1-, 2-, 3-hydroxypropyl, 1,3-dihydroxy-2-propyl, 1,3-dihydroxybutyl, 1,2,3,4,5,6-hexahydroxy-2-hexyl.

As used herein, “nucleophile” refers to a nucleophilic agent wherein a negatively charged carbon, oxygen or nitrogen anion is associated with a metal counter-ion. Examples include, but are not limited to, those agents known in the art of organic synthesis as Grignard reagents, cuprates, alkyl metals, and the like.

The coupling reaction is preferably conducted in the presence of a catalyst and a base. A catalyst is a chemical substance that in small quantities notably accelerates the rate of a chemical reaction while itself remaining essentially unchanged. Generally speaking, catalysts are specific in activity toward various types of chemical reactions such as alkylation, condensation, oxidation, and polymerization. The most preferred bases for use in the present method are (1) any alkali metal hydroxide carbonate, bicarbonate, phosphate, or alkoxide, or (2) any tertiary organic amine, or (3) mixtures of (1) and (2).

The coupling reaction requires the presence of a “base” which is an agent, capable of accepting a hydrogen atom from an acidic hydrogen donor agent. Examples of such bases include, but are not limited to, organic bases such as aromatic amines such as pyridine, N,N-diethylaniline; aliphatic amines including, but not limited to, trialkyl amines such as triethylamine, N-methylmorpholine (NMM), N,N-diisopropylethylamine, N,N-diethylcyclohexylamine, N,N-dimethylcyclohexylamine, N,N,N′-triethylenediamine, N,N-dimethyloctylamine; 1,5-diazabicyclo[4.3.0]non-5-ene (DBN); 1,4-diazabicyclo[2.2.2]octane (DABCO); 1,8-diazabicyclo[5.4.0]undec-7-ene (DBU); tetramethylethylenediamine TMEDA); and substituted pyridines such as N,N-dimethylaminopyridine (DMAP), 4-pyrrolidinopyridine, and 4-piperidinopyridine. Additionally, suitable bases can be selected from polymeric tertiary amines, as well as polymeric aromatic amines. Examples of strong bases include, but are not limited to, alkyllithiums such as isobutyllithium, n-hexyllithium, n-octyllithium, n-butyllithium, s-butyllithium, t-butyllithium, phenyllithium, and triphenylmethyllithium; metal amides such as sodium amide, potassium amide, and lithium amide; metal hydrides such as sodium hydride, potassium hydride, and lithium hydride; and metal dialkylamides such as sodium and potassium salts of methyl, ethyl, n-propyl, i-propyl, n-butyl, t-butyl, trimethylsilyl, and cyclohexyl substituted amides. Other examples of strong bases include, but are not limited to, alkyl magnesium halides and aryl magnesium halides such as, methyl magnesium chloride, ethyl magnesium chloride, propyl magnesium chloride, n-butyl-, iso-butyl-, or t-butylmagnesium chloride, pentyl magnesium chloride, hexyl magnesium chloride, and phenyl magnesium chloride. Preferred strong bases are n-butyl magnesium chloride and phenyl magnesium chloride. “Aqueous base” refers to bases that are water soluble, and useful for neutralizing aqueous acids. Examples of such bases include, but are not limited to aqueous solutions of: sodium, lithium, and potassium salts of carbonates; sodium, lithium, and potassium salts of bicarbonates; and sodium, lithium and potassium salts of hydroxides.

The present method preferably uses an organometallic catalyst compound having the formula QM wherein M is an element selected from the group consisting of palladium, platinum, rhodium, and nickel and Q is an organic ligand. Preferred organic ligands include triphenyl-phosphine, tris(2-methoxyphenyl)phosphine, acetate, dibutylamine-C₆H₆, and n-propyl-Cl. The most preferred catalyst is tetrakis(triphenylphosphine)palladium, which may be used as supplied or prepared in situ in accordance with the methods know in the art.

The reaction of the methods claimed herein are carried out in suitable solvents which may be readily selected by one skilled in the art of organic synthesis, the suitable solvents generally being any solvent which is substantially non-reactive with the starting materials (reactants), the intermediates, or products at the temperatures at which the reactions are carried out, i.e., temperatures which may range from the solvent's freezing temperature to the solvent's boiling temperature. A given reaction may be carried out in one solvent or a mixture of more than one solvent. Depending on the particular reaction, suitable solvents for a particular reaction or work-up following the reaction may be selected. Such suitable solvents, as used herein may include, by way of example and without limitation, hydrocarbon solvents, ether solvents, and polar aprotic solvents.

Suitable hydrocarbon solvents include, but are not limited to benzene, cyclohexane, pentane, hexane, toluene, cycloheptane, methylcyclohexane, heptane, ethylbenzene, m-, o-, or p-xylene, octane, indane, and nonane.

Suitable ether solvents include, but are not limited to dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol diisopropyl ether, anisole, and t-butyl methyl ether.

Suitable polar aprotic solvents include, but are not limited to dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DNI), N-methylpyrrolidinone (NMP), formamide, N-methylacetamide, N-methylformamide, acetonitrile (ACN), dimethylsulfoxide, propionitrile, ethyl formate, methyl acetate, hexachloroacetone, acetone, ethyl methyl ketone, ethyl acetate, isopropyl acetate, t-butyl acetate, sulfolane, N,N-dimethylpropionamide, nitromethane, nitrobenzene, and hexamethylphosphoramide. The preferred solvent system comprises the polar aprotic system, and the most preferred solvent is DMF.

Aqueous solvents comprising mixtures of water and either alcohols, such as methanol or ethanol, or polar aprotic solvents, such as ethers, such as methyl ethyl ether, may be used, but are not preferred to achieve the benefits of high yield and product purity available by the claimed methods.

The method of present invention preferably uses an R₂-substituted aromatic boronic acid wherein R₂ is an alkyl, alkoxy, alkenyl, cycloalkyl, cycloalkenyl, aralkyl, aryl, carbonylalkyl, amino, alkylamino, dialkylamino, hydroxyl, hydroxyalkyl, nitro, cyano, isocyanato, carbamyl, amido, alkylamido, dialkylamido, trifluoromethyl, or aryloxy group.

The preferred aromatic boronic acids compound comprises aromatic groups that are preferably substituted or unsubstituted phenyl, biphenyl, triphenyl, naphthyl, phenylnaphthyl, thienyl, furyl, pyrrolyl, and/or pyridyl.

The present invention may be further described as a method for preparing carboxyl substituted polyaryl compounds by a reaction comprising the cross-coupling of a substituted aromatic boronic acid or borate of formula II 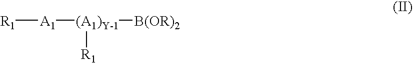
with a halo-substituted aromatic carboxylic acid of formula IlI, and/or a salt thereof, 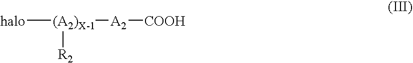
in the presence of a base and a palladium catalyst yielding a carboxyl substituted polyaryl compound of formula I, and/or a salt thereof, 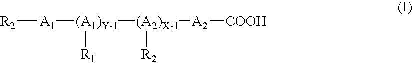
wherein

A₁ and A₂ are each independently phenyl, biphenyl, triphenyl, naphthyl, phenylnaphthyl, pyridyl, pyrrolyl, thienyl, furyl, or pyridyl;

R is independently hydrogen, lower alkyl or together consists of alkylene to form a cyclic boronic acetal;

R₁ and R₂ are independently alkyl, alkoxy, alkenyl, cycloalkyl, cycloalkenyl, aralkyl, carbonylalkyl, aryl, amino, alkylamino, dialkylamino, hydroxyl, hydroxyalkyl, nitro, cyano, isocyanato, amido, alkylamido, dialkylamido, trifluoromethyl, or aryloxy; and

X and Y are independently 1 to about 10.

A particularly preferred halo group in formula (III) is iodo or bromo.

A special embodiment of the present method prepares compounds of formula (I) above wherein both A₁ and A₂ are independently substituted or unsubstituted phenyl groups.

A further aspect of the present invention is the ability to prepare polyaryl, more particularly, polyphenyl compounds in a chain where the phenyl ring orientation is chosen for each member ring as the series, and substituent groups thereon may also be selected for their relative orientation to the phenyl-phenyl carbon bonds. For example, the R₂ substituents may be attached to the phenyl in an ortho, meta, or para position relative to the phenyl-phenyl bond.

One class of boronic compounds may be described in accordance with the following formula:

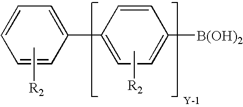

Another class of boronic acids is described as follows:

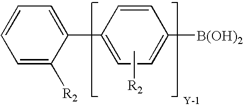

Another class of boronic acids is described as follows:

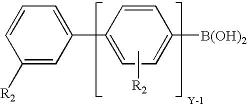

Yet another class of boronic acids is described as follows:

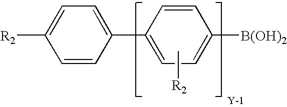

A particular embodiment of a preferred class of boronic acids is described by the following formula:

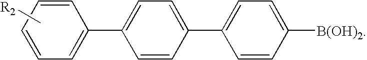

Another embodiment of a preferred class of boronic acids is described by the following formula:

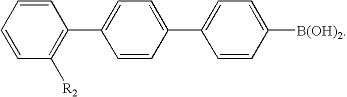

Yet another particular embodiment of a preferred class of boronic acids is described by the following formula:

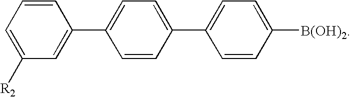

Yet another particular embodiment of a preferred class of boronic acids is described by the following formula:

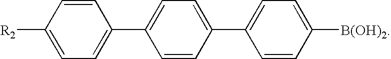

A particularly preferred embodiment of boronic acids is described by the following formula:

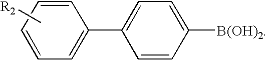

A particularly preferred subclass of preferred embodiment of boronic acids is described by the following formula:

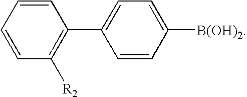

Another particularly preferred subclass of preferred embodiment of boronic acids is described by the following formula:

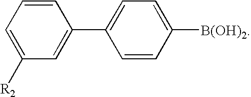

Another particularly preferred subclass of preferred embodiment of boronic acids is described by the following formula:

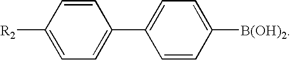

Particularly preferred subclasses of preferred embodiments of boronic acids are described by the following formulae:

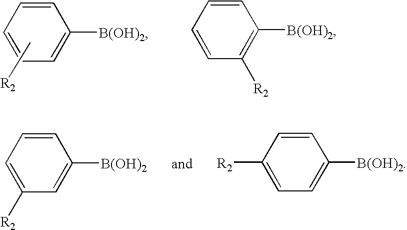

The boronic acids or borates useful in the present method may be prepared by treating a 1-halo-substituted aryl or polyaryl compound with magnesium to form the corresponding aryl magnesium halide followed by treating the aryl magnesium halide with trimethylborate to form aryl or polyaryl boronic acid.

The present invention is further described by reference to the following example.

### EXAMPLE

### 4″-n-Pentyloxy-1′:4′1″-Terphenyl-4-Carboxylic Acid

Step I. Preparation of 4-pentyloxyphenyl boronic acid (I)

To 100 ml of anhydrous methyl isobutyl ether at −80 C. under nitrogen is added a THF solution of 4-pentyloxyphenylmagnesium bromide (40.1 g, 150mmol) and trimethylborate (16.2 g,156mmol). After stirring the reaction mixture for 5 hours, the reaction is quenched with 50 ml of water giving a liquid phase and a gel-like solid phase. After holding the mixture for a period of 24 hours at room temperature, an additional 60 ml of water is added followed by 15 ml of conc. hydrochloric acid. The reaction mixture separates into a light brown aqueous layer and a yellow colored organic top layer. The top layer is decanted, and washed three times with aqueous sodium hydroxide (4%) using a total of 7.8 g ( 0.195 mole) of sodium hydroxide. The mixture separates into a top clear liquid layer which is disposed of and a bottom aqueous layer which is washed with hexane resulting in a clear, colorless layer and an orange colored aqueous bottom layer. The bottom aqueous layer is washed and then stirred with hexane yielding a white solid that is filtered and rinsed twice with hexane leaving wet solids in the form of shiny crystals. A slurry of the solid in water (pH>10) is stirred with conc. hydrochloric acid for 36 hours, then filtered and rinsed with water. The wet solids are dried by azeotropic distillation with hexane, cooled and filtered giving shiny, fibrous crystals of the title compound with a HPLC retention time of 2.259 min and a melting point of 112-115 C.

Step II The preparation of 4″-n-pentyloxy-1′:4′1″-terphenyl-4-carboxylic acid (II)

A mixture of boronic acid (I) (2.18 g 10.5 mmol) prepared in step I above and 4′-bromo-4-biphenyl carboxylic acid (2.77 g, 10 mmol) is suspended in 20 g of DMF and heated under nitrogen to 95 C. forming a clear solution. To the heated solution at 89 C. is added Pd(OAc)₂ (0.067 g., in 2 mL DMF), triphenylphosphine (0.23 g) and triethylamine (3 g) The mixture is stirred at 90-105 C. for 32 hours during which period a solid phase appears which is separated by filtration, suspended in THF (30 g), and heated under reflux for 1 hour. An aqueous solution of KCl (10 g, 9.8%) is then added and heating resumed for 20 min. The resulting slurry is filtered and the wet cake returned to fresh THF and then heated under reflux followed by the addition of aqueous solution of KCl as in the preceding step. The resulting filter cake is washed with THF for an extended period at room temperature and then 10 g water is added and the mixture heated under reflux for 20 minutes. Solids are filtered and rinsed with THF yielding 0.8 g of the title product.

The compounds described herein may have asymmetric centers. Unless otherwise indicated, all chiral, diastereomeric and racemic forms are included in the present invention. Many geometric isomers of olefins, C═N double bonds, and the like can also be present in the compounds described herein, and all such stable isomers are contemplated in the present invention. It will be appreciated that compounds of the present invention that contain asymmetrically substituted carbon atoms may be isolated in optically active or racemic forms. Methods on how to prepare optically active forms from optically active starting materials are known in the art, such as by resolution of racemic forms or by synthesis. All chiral, diastereomeric, racemic forms and all geometric isomeric forms of a structure are intended.

The present invention includes all isotopes of atoms occurring in the intermediates or final compounds. Isotopes include those atoms having the same atomic number but different mass numbers. By way of general example and without limitation, isotopes of hydrogen include tritium and deuterium; isotopes of carbon include ¹³C and ¹⁴C.

The present invention is contemplated to be practiced on at least a multigram scale, kilogram scale, multikilogram scale, or industrial scale. Multigram scale, as used herein, is preferably the scale wherein at least one starting material is present in 10 grams or more, more preferably at least 50 grams or more, even more preferably at least 100 grams or more. Multikilogram scale, as used herein, is intended to mean the scale wherein more than one kilogram of at least one starting material is used. Industrial scale as used herein is intended to mean a scale which is other than a laboratory scale and which is sufficient to supply product sufficient for either clinical tests or distribution to consumers.

1. A method for the preparation of an polyaromatic carboxylic acid compound and/or salt thereof comprising reacting an aromatic boronic acid with a halo-substituted, aromatic carboxylc acid compound and/or salt thereof.

2. The method of claim 1 wherein the aromatic boronic acid is R₂-substituted wherein R₂ is independently alkyl, alkoxy, alkenyl, cycloalkyl, cycloalkenyl, aralkyl, carbonylalkyl, amino, alkylamino, dialkylamino, hydroxyl, hydroxyalkyl, nitro, cyano, isocyanato, carbamyl, amido, alkylamido, dialkylamido, trifluoromethyl or aryloxy.

3. The method of claim 2 wherein said reaction is conducted in the presence of a catalyst and a base.

4. The method according to claim 3 wherein said catalyst is an organometallic catalyst compound having the formula QM wherein M is an element selected from the group consisting of palladium, platinum, rhodium, and nickel and Q is an organic ligand.

5. The method according to claim 4 wherein said organic ligand is selected from the group consisting of triphenylphosphine, tris(2-methoxyphenyl)phosphine, acetate, dibutylamine-C₆H₆, and n-propyl-Cl.

6. The method according to claim 1 wherein said aromatic compound comprises substituted phenyl, biphenyl, triphenyl, naphthyl, phenylnaphthyl, thienyl, furyl, pyrrolyl, pyridyl.

7. The method of claim 1 wherein said halo-substituent is iodo or bromo.

8. The method according to claim 4 wherein said organo metallic compound is tetrakis(triphenylphosphine)palladium.

9. A method for the preparation of an R₁, R₂ substituted polyaromatic compound of formula I, and/or a salt thereof, 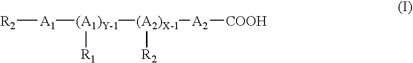
comprising reacting an aromatic boronic acid of formula II 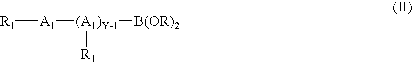
with a halo-substituted aromatic compound of formula III, and/or a salt thereof, 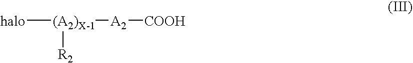
wherein
A₁ and A₂ are each independently phenyl, biphenyl, triphenyl, naphthyl, phenylnaphthyl, pyridyl, pyrrolyl, thienyl, furyl, or pyridyl.
R₁ and R₂ are independently alkyl, alkoxy, alkenyl, cycloalkyl, cycloalkenyl, aralkyl, carbonylalkyl, aryl, amino, alklamino, dialkylamino, hydroxyl, hydroxyalkyl, nitro, cyano, isocyanato, amido, alkylamido, dialkylamido, trifluoromethyl, or aryloxy;
Y is 1 to about 10;
X is 1 to about 10; and
R₂ is independently hydrogen, lower alkyl or together consists of alkylene to form a cyclic boronic acetal.

10. The method of claim 9 where A₁ is a phenyl group and A₂ is a phenyl group.

11. The method of claim 3 wherein said base is (1) any alkali metal hydroxide carbonate, bicarbonate, phosphate, or alkoxide, or (2) any tertiary organic amine, or (3) mixtures of (1) and (2).

12. The method of claim 10 where in R₂ is attached to the phenyl in an ortho, meta, or para position.

13. The method of claim 9 where in (R₂-A₁)_{Y}-B(OH)₂ is

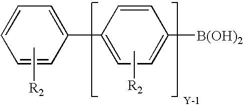

14. The method of claim 13 wherein (R₂-A₁)_{Y}-B(OH)₂ is

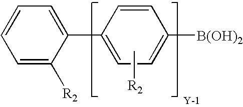

15. The method of claim 13 wherein (R₂-A₁)_{Y}-B(OH)₂ is

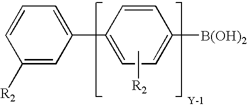

16. The method of claim 13 wherein (R₂-A₁)_{Y}-B(OH)₂ is

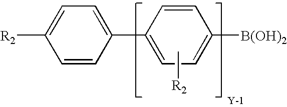

17. The method of claim 13 wherein (R₂-A₁)_{Y}-B(OH)₂ is

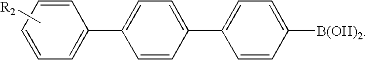

18. The method of claim 14 wherein (R₂-A₁)_{Y}-B(OH)₂ is

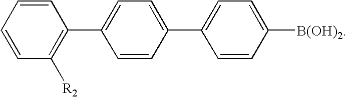

19. The method of claim 15 wherein (R₂-A₁)_{Y}-B(OH)₂ is

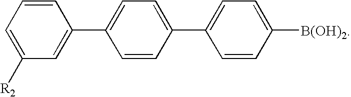

20. The method of claim 16 wherein (R₂-A₁)_{Y}-B(OH)₂ is

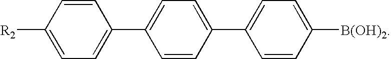

21. The method of claim 14 wherein (R₂-A₁)_{Y}-B(OH)₂ is

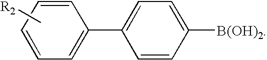

22. The method of claim 14 wherein (R₂-A₁)_{Y}-B(OH)₂ is

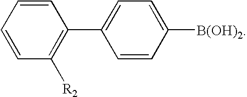

23. The method of claim 15 wherein (R₂-A₁)_{Y}-B(OH)₂ is

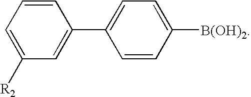

24. The method of claim 16 wherein (R₂-A₁)_{Y}-B(OH)₂ is

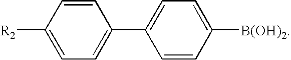

25. The method of claim 13 wherein (R₂-A₁)_{Y}-B(OH)₂ is

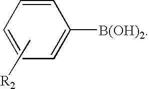

26. The method of claim 14 wherein (R₂-A₁)_{Y}-B(OH)₂ is

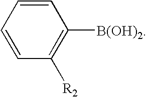

27. The method of claim 15 wherein (R₂-A₁)_{Y}-B(OH)₂ is

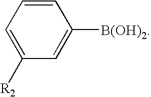

28. The method of claim 16 wherein (R₂-A₁)_{Y}-B(OH)₂ is

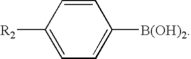

29. The method of claim 16 wherein R₂-(A₁)_{Y}-B(OH)₂ is selected from the group consisting of

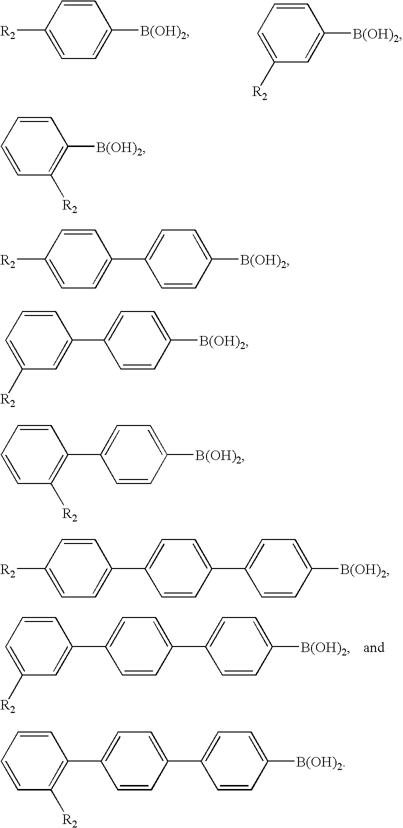

30. A method according to claim 24, for the preparation of 4″alkyloxy-1′: 4′1″-terphenyl-4-carboxylic acid comprising the step of reacting 4-alkyloxyphenyl boronic acid with 4′-halo-4-biphenyl carboxylic acid.

31. The method of claim 30 wherein the preparation further comprises the step of treating 1-halo-4-alkyloxybenzene with magnesium to form 4-alkyloxyphenylmagnesium halide.

32. The method of claim 31 wherein the preparation further comprises the step of treating a 4-alkyloxyphenylmagnesium halide with trimethylborate to form 4-alkylloxyphenyl boronic acid.

33. The method of claim 32 wherein the alkyl is n-pentyl.
